(19) United States
(12) Patent Application Publication
(10) Pub. No.: US 2026/0092806 A1
(43) Pub. Date: Apr. 2, 2026

(54) Systems and Methods for Generating Synthetic Cardio-Respiratory Signals

(71) Applicant: Sleep Number Corporation, Minneapolis, MN (US)

(72) Inventors: Omid Sayadi, San Jose, CA (US); Steven Jay Young, Los Gatos, CA (US)

(21) Appl. No.: 19/288,157

(22) Filed: Aug. 1, 2025

Related U.S. Application Data
(63) Continuation of application No. 18/094,751, filed on Jan. 9, 2023, now abandoned.
Application No. 18/094,751 is a continuation of application No. 16/777,385, filed on Jan. 30, 2020, now abandoned.
Application No. 16/777,385 is a continuation-in-part of application No. 16/595,848, filed on Oct. 8, 2019, now abandoned.
(60) Provisional application No. 62/804,623, filed on Feb. 12, 2019.
(60) Provisional application No. 62/804,623, filed on Feb. 12, 2019.

(51) Int. Cl. G01G 19/44; A47C 19/02; A47C 19/22; A61B 5/00; A61B 5/0205; A61B 5/08; A61B 5/11; G01G 19/52; G01G 21/02; G01V 9/00; G05B 15/02; G06N 5/04; G06N 20/00; G08B 21/22

(52) U.S. Cl. CPC G01G 19/445; A47C 19/027; A47C 19/22; A61B 5/0205; A61B 5/1102; A61B 5/1115; A61B 5/4818; A61B 5/6891; A61B 5/6892; A61B 5/7203; A61B 5/7246; A61B 5/725; A61B 5/7267; A61B 5/7278; A61B 5/7282; A61B 5/7415; G01G 19/52; G01G 21/02; G01V 9/00; G05B 15/02; G06N 5/04; G06N 20/00; G08B 21/22; A61B 5/0816; A61B 2560/0223

(57) ABSTRACT

Devices and methods for generating synthetic cardio-respiratory signals from one or more ballistocardiogram (BCG) sensors. A method for determining item specific parameters includes obtaining ballistocardiogram (BCG) data from one or more sensors, where the one or more sensors capture BCG data for one or more subjects in relation to a substrate. For each subject, the captured BCG data is pre-processed to obtain cardio-respiratory BCG data. The cardio-respiratory BCG data is sub-sampled to generate the cardio-respiratory BCG data at a cardio-respiratory sampling rate conducive to cardio-respiratory signal generation. The sub-sampled cardio-respiratory BCG data is cardio-respiratory processed to generate a cardio-respiratory parameter set. A synthetic cardio-respiratory signal is generated from at least the cardio-respiratory parameter set and a cardio-respiratory event morphology template. A condition of the subject is determined based on the synthetic cardio-respiratory signal.

### CROSS-REFERENCE TO RELATED APPLICATIONS

This application is a continuation of U.S. patent application Ser. No. 18/094,751, filed Jan. 9, 2023, which is a continuation of U.S. patent application Ser. No. 16/777,385, filed on Jan. 30, 2020 (abandoned), which is a continuation-in-part of U.S. patent application Ser. No. 16/595,848, filed Oct. 8, 2019 (abandoned), which claims priority to U.S. Provisional Application Patent Ser. No. 62/804,623, filed Feb. 12, 2019, the entire disclosures of which are hereby incorporated by reference.

This application claims priority to and the benefit of U.S. Provisional Application Patent Ser. No. 62/804,623, filed Feb. 12, 2019, the entire disclosure of which is hereby incorporated by reference.

### TECHNICAL FIELD

This disclosure relates to systems and methods for determining and monitoring biosignals, such as cardiac and respiratory biosignals, based on contactless sensor signals.

### BACKGROUND

Cardiac and respiratory signal monitoring requires electrical equipment and sensors connected to a subject using wires, belts, nasal cannula, or like attachments. These attachments limit the mobility of the subject and cannot be conveniently done for long hours, especially in non-hospital type settings like a home. Moreover, the need to re-attach the sensors limit the repeatability and consistency of measurements over long periods of time.

### SUMMARY

Disclosed herein are implementations of devices and methods for generating synthetic cardio-respiratory signals from one or more ballistocardiogram (BCG) sensors.

In an implementation, a method for determining item specific parameters includes obtaining ballistocardiogram (BCG) data from one or more sensors, where the one or more sensors capture BCG data for one or more subjects in relation to a substrate. For each subject, the captured BCG data is pre-processed to obtain cardio-respiratory BCG data. The cardio-respiratory BCG data is sub-sampled to generate the cardio-respiratory BCG data at a cardio-respiratory sampling rate conducive to cardio-respiratory signal generation. The sub-sampled cardio-respiratory BCG data is cardio-respiratory processed to generate a cardio-respiratory parameter set. A synthetic cardio-respiratory signal is generated from at least the cardio-respiratory parameter set and a cardio-respiratory event morphology template. A condition of the subject is determined based on the synthetic cardio-respiratory signal.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is an illustration of a bed incorporating sensors as disclosed herein.

FIG. 2 is an illustration of the bed frame with sensors incorporated, the bed frame configured to support a single subject.

FIG. 3 is an illustration of a bed frame with sensors incorporated, the bed frame configured to support two subjects.

FIG. 4 is a system architecture for a multidimensional multivariate multiple sensors system.

FIG. 5 is a processing pipeline for obtaining sensors data.

FIG. 6 is a pre-processing pipeline for processing the sensors data into multiple sensors multiple dimensions array (MSMDA) data.

FIG. 7 is a flowchart for generating synthetic cardio-respiratory signals.

FIG. 8 is an example of signals generated in the method of generating synthetic cardio-respiratory signals.

FIG. 9 is a flowchart for generating synthetic cardio signals.

FIG. 10 is a flowchart for generating synthetic respiratory signals.

FIG. 11 is a flowchart for generating synthetic cardio-respiratory time series and sound streams using a defined template sound.

FIG. 12 is a flowchart for generating synthetic cardio-respiratory time series and sound streams using an adaptive template sound.

FIG. 13A is a flowchart for generating synthetic cardio-respiratory signals from a multiple sensor multiple dimensions system.

FIGS. 13B-E are example surface location maps for a multidimensional multivariate multiple sensors system with 4 sensors.

FIG. 14 is an example of surface location map and spatial cardio-respiratory maps.

FIG. 15 is a swim lane diagram for building and classifying individualized and population morphology templates.

### DETAILED DESCRIPTION

Disclosed herein are systems and methods for generating and monitoring biosignal weight, morphology, rhythm and rate information of a subject from one or more ballistocardiogram (BCG) sensors. The systems and methods use one or more patient non-contact sensors such as pressure, load, weight, force, motion, vibration or accelerometer based sensors to continuously capture the mechanical vibrations of the body, heart, and lungs and translate that into synthetic cardio-respiratory signals. In particular, the systems and methods enable contactless generation of synthetic electrocardiograma respiratory signals. In addition, cardiac and respiratory audio streams may be generated for playback with the synthetic electrocardiogramd respiratory signals.

The mechanical vibrations are transformed into synthetic signals that look like the electrical measurements from the heart or the flow/ventilation measurements from the lungs. The mechanical vibrations are also transformed into synthetic signals that sound like the mechanical movements from the heart or the flow/ventilation movements from the lungs. The synthetic signals along with synthetic cardiac and breathing audio streams, can be stored, displayed onsite, displayed remotely, or analyzed using automated processing or artificial intelligence (AI) techniques.

The synthetic signals, the synthetic cardiac and breathing audio streams, or combinations thereof can be used to monitor and detect a variety of physiological conditions. In an implementation, the synthetic signals can be used to detect physiological conditions that impact the rhythm and rate of the biosignals, including atrial fibrillation, atrial flutter, ventricular fibrillation, ventricular flutter, bundle branch blocks, valve stenosis, myocardial ischemia, supraventricular tachycardia, apnea, hypopnea, Cheyne stoke breathing, snoring and the like. In an implementation, the synthetic audio streams can be used to detect physiological conditions that impact the sound of the biosignals, including heart murmurs, snoring, coughing, wheezing, rales, rhonchi, and the like.

In an implementation, the system generates cardiac beat morphology or respiratory breath morphology templates as a baseline for a subject which can be monitored in real-time or over a period of time to identify changes from the subject's baseline. In an implementation, the cardiac beat morphology template can be used to predict or detect heart conditions that alter the cardiac morphology such as, but not limited to, atrial fibrillation, atrial flutter, ventricular fibrillation, ventricular flutter, bundle branch blocks, valve stenosis, myocardial ischemia, and the like. In an implementation, the respiratory breath morphology template can be used to predict or detect breathing conditions that alter the respiratory morphology such as, but not limited to, apnea, hypopnea, Cheyne stoke breathing, snoring, and the like.

A database of cardiac beat morphology and/or respiratory breath morphology templates stored can be used to build individualized and population models of normal morphologies and different diseases and use those models to train machine learning classifiers to automatically detect changes in morphologies due to arrhythmias or diseases.

In an implementation using multiple sensors, the system can create spatial cardiac and respiration maps which provide different views of the heart and lungs function including a complete three-dimensional view of the electrical activity of the heart and mechanical activity of the lungs. Spatial maps can be used to diagnosis conditions that affect a localized portion of the heart or lung, such as myocardial infarction for example, that would otherwise be missed or undiagnosed. The spatial maps can be used to predict and diagnose changes in health status, arrhythmias, and diseases related to, for example, cardio-respiratory conditions.

The availability of multiple sensors enables the generation of a fuller picture of the three-dimensional electrical activity of the heart and lung. The body including the heart and lung are is a three-dimensional structure, and the electrical currents and respiratory pathways are spread out in all directions across the body. The more points of data that are recorded, the more accurate the representation of the electrical and respiratory activity. The combination of surface location maps for multiple sensors and the spatial cardiac or respiratory map(s) enables the diagnosis of conditions that affect one localized portion of the heart or lung, such as for example, myocardial infarction.

In an implementation, the one or more sensors can be any sensor that records at least one ballistocardiogram (BCG) signal using non-contact sensors such as pressure, load, force, motion or accelerometer. The contactless signals can be obtained from one or more sensors that are implemented in a variety of forms or structures including, but not limited to, bed, couch, chair, examination table, floor, air chamber bed, wearable clothing, smart scale, and the like. The one or more sensors can be configured in any type of surface depending on the application.

The data collected by the sensors can be collected for a particular subject for a period of time, or indefinitely, and can be collected in any location, such as at home, at work, in a hospital, nursing home or other medical facility. A limited period of time may be a doctor's visit to assess biometric data against baseline data or can be for a hospital stay to monitor cardiac signals for atrial fibrillation patterns. Messages can be sent to family and caregivers and/or reports can be generated for doctors.

The data collected by the sensors can be collected and analyzed for much longer periods of time, such as years or decades, when the sensors are incorporated into a subject's personal or animal's residential bed. The sensors and associated systems and methods can be transferred from one substrate to another to continue to collect data from a particular subject.

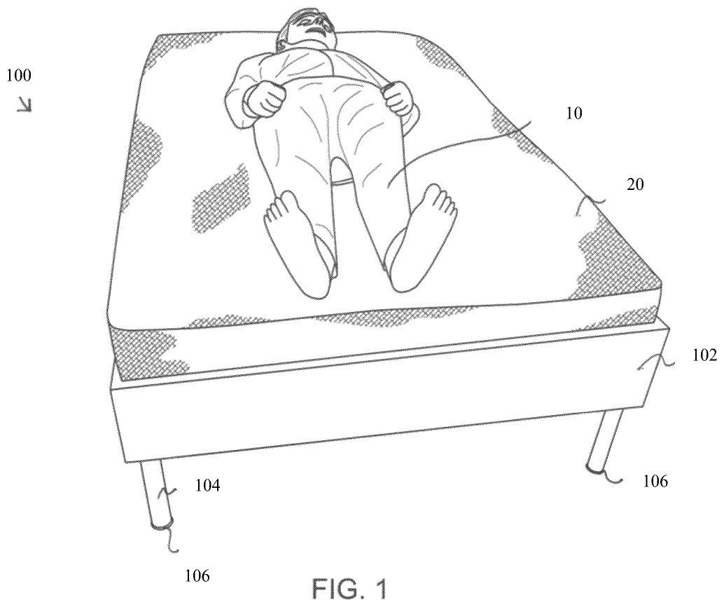

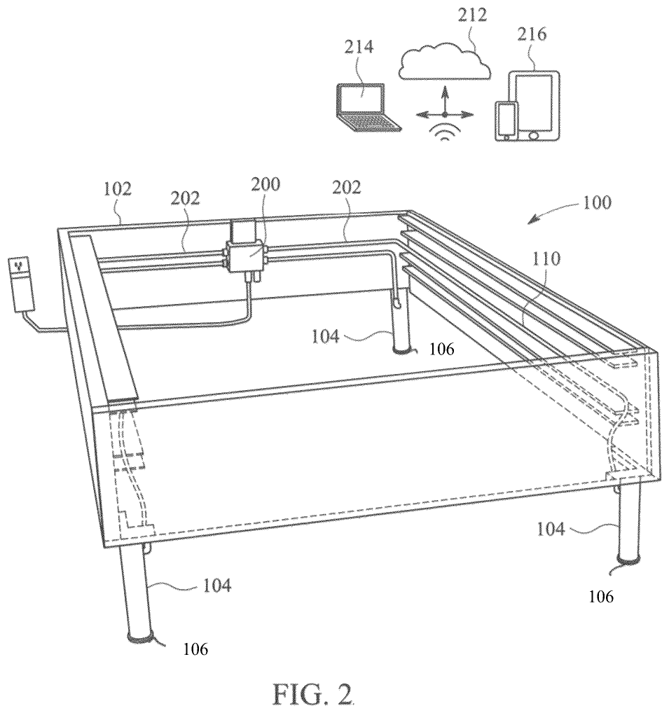

FIGS. 1 and 2 illustrate a system 100 for measuring data specific to a subject 10 using gravity. The system 100 can comprise a substrate 20 on which the subject 10 can lie. The substrate 20 is held in a frame 102 having multiple legs 104 extending from the frame 102 to a floor to support the substrate 20. Multiple load or other sensors 106 can be used, each load or other sensor 106 associated with a respective leg 104. Any point in which a load is transferred from the substrate 20 to the floor can have an intervening load or other sensor 106. Placement of the sensors is illustrative and they can be located in a variety of locations including, but not limited to, bed frame, mattress, and the like.

As illustrated in FIG. 2, a local controller 200 can be wired or wirelessly connected to the load or other sensors 106 and collects and processes the signals from the load or other sensors 106. The controller 200 can be attached to the frame 102 so that it is hidden from view, can be on the floor under the substrate or can be positioned anywhere a wireless transmission can be received from the load or other sensors 106 if transmission is wireless. Wiring 202 may electrically connect the load or other sensors 106 to the controller 200. The wiring 202 may be attached to an interior of the frame 102 and/or may be routed through the interior channels 110 of the frame 102. The controller 200 can collect and process signals from the load or other sensors 106. The controller 200 may also be configured to output power to the sensors and/or to printed circuit boards disposed in the load or other sensors 106.

The controller 200 can be programmed to control other devices based on the processed data, such as bedside or overhead lighting, door locks, electronic shades, fans, etc., the control of other devices also being wired or wireless. Alternatively, or in addition to, a cloud based computer 212 or off-site controller 214 can collect the signals directly from the load or other sensors 106 for processing or can collect raw or processed data from the controller 200. For example, the controller 200 may process the data in real time and control other local devices as disclosed herein, while the data is also sent to the off-site controller 214 that collects and stores the data over time. The controller 200 or the off-site controller 214 may transmit the processed data off-site for use by downstream third parties such a medical professionals, fitness trainers, family members, etc. The controller 200 or the off-site controller 214 can be tied to infrastructure that assists in collecting, analyzing, publishing, distributing, storing, machine learning, etc. Design of real-time data stream processing has been developed in an event-based form using an actor model of programming. This enables a producer/consumer model for algorithm components that provides a number of advantages over more traditional architectures. For example, it enables reuse and rapid prototyping of processing and algorithm modules. As another example, data streams can be enabled/disabled dynamically and routed to or from modules at any point within a group of modules comprising an algorithmic system, enabling computation to be location-independent (i.e., on a single device, combined with one or more additional devices or servers, on a server only, etc.).

The long-term collected data can be used in both a medical and home setting to learn and predict patterns of sleep, illness, etc. for a subject. As algorithms are continually developed, the long-term data can be reevaluated to learn more about the subject. Sleep patterns, weight gains and losses, changes in heart beat and respiration can together or individually indicate many different ailments. Alternatively, patterns of subjects who develop a particular ailment can be studied to see if there is a potential link between any of the specific patterns and the ailment.

The data can also be sent live from the controller 200 or the off-site controller 214 to a connected device 216, which can be wirelessly connected for wired. The connected device 216 can be, as examples, a mobile phone or home computer. Devices can subscribe to the signal, thereby becoming a connected device 216.

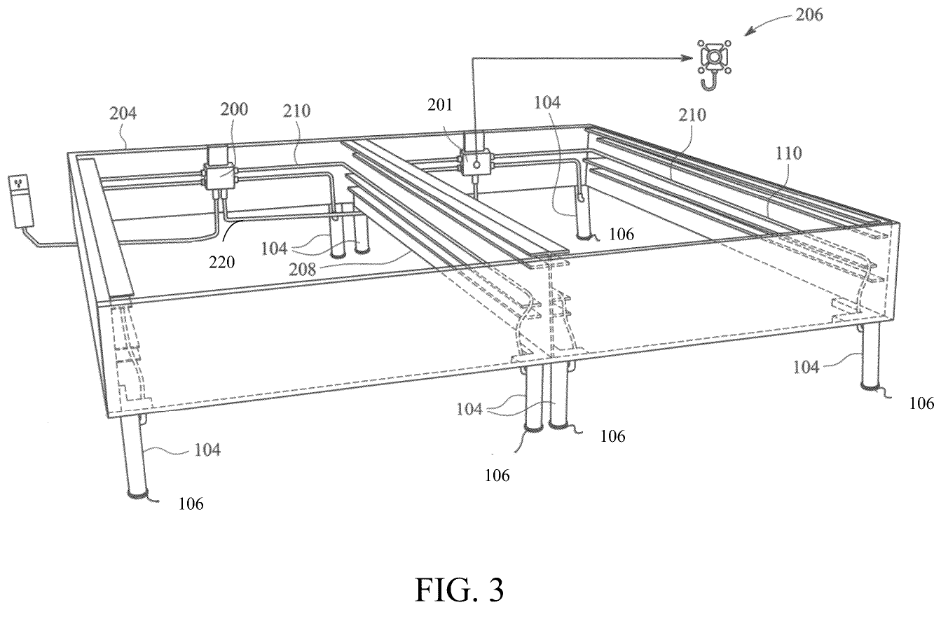

FIG. 3 is a top perspective view of a frame 204 for a bed 206 used with a substrate on which two or more subjects can lie. The bed 206 may include features similar to those of the bed 100 except as otherwise described. The bed 206 includes a frame 204 configured to support two or more subjects. The bed 206 may include eight legs, including one load or other sensor 106 disposed at each leg 104. In other embodiments, the bed may include nine legs 104 and nine load or other sensors 106, the additional sensor 106 disposed at the middle of the central frame member 208. In other embodiments, the bed 206 may include any arrangement of load or other sensors 106. Two controllers 200 and 201, for example, can be attached to the frame 204. The controllers 200 may be in wired or wireless communication with its respective sensors and optionally with each other. Each of the controllers 200 collects and processes signals from a subset of load or other sensors 106. For example, one controller 200 can collect and process signals from load or other sensors 106 (e.g. four load or other sensors) configured to support one subject lying on the bed 206. Another controller 200 can collect and process signals from the other load or other sensors 106 (e.g. four load or other sensors) configured to support the other subject lying on the bed 206. Wiring 210 may connect the load or other sensors 106 to either or both of the controllers 200 attached to the frame 204. In an implementation, wiring 220 can connect controllers 200 and 201. The wiring 210 may also connect the controllers 200. In other embodiments, the controllers may be in wireless communication with each other. In an implementation, one of the controllers 200 and 201, can process the signals collected by both of the controllers 200 and 201.

Examples of data determinations that can be made using the systems herein are described. The algorithms use the number of sensors and each sensor's angle and distance with respect to the other sensors. This information is predetermined. Software algorithms will automatically and continuously maintain a baseline weight calibration with the sensors so that any changes in weight due to changes in a mattress or bedding is accounted for.

The load or other sensors herein utilize macro signals and micro signals and process those signals to determine a variety of data, described herein. Macro signals are low frequency signals and are used to determine weight and center of mass, for example. The strength of the macro signal is directly influenced by the subject's proximity to each sensor.

Micro signals are also detected due to the heartbeat, respiration and to movement of blood throughout the body. Micro signals are higher frequency and can be more than 1000 times smaller than macro signals. The sensors detect the heart beating and can use its corresponding amplitude or phase data to determine where on the substrate the heart is located, thereby assisting in determining in what location, angular orientation, and body position the subject is laying as described and shown herein. In addition, the heart pumps blood in such a way that it causes top to bottom changes in weight. There is approximately seven pounds of blood in a human subject, and the movement of the blood causes small changes in weight that can be detected by the sensors. These directional changes are detected by the sensors. The strength of the signal is directly influenced by the subject's proximity to the sensor. Respiration is also detected by the sensors. Respiration will be a different amplitude and a different frequency than the heart beat and has different directional changes than those that occur with the flow of blood. Respiration can also be used to assist in determining the exact location, angular orientation, and body position of a subject on the substrate. These bio-signals of heart beat, respiration and directional movement of blood are used in combination with the macro signals to calculate a large amount of data about a subject, including the relative strength of the signal components from each of the sensors, enabling better isolation of a subject's bio-signal from noise and other subjects.

As a non-limiting example, the cardiac bio-signals in the torso area are out of phase with the signals in the leg regions. This allows the signals to be subtracted which almost eliminates common mode noise while allowing the bio-signals to be combined, increasing the signal to noise by as much as a factor of 3 db or 2× and lowering the common or external noise by a significant amount. By analyzing the phase differences in the 1 Hz to 10 Hz range (typically the heart beat range) the body position of a person laying on the bed can be determined. By analyzing the phase differences in the 0 to 0.5 Hz range, it can be determined if the person is supine, prone or laying on their side, as non-limiting examples.

Because signal strength is still quite small, the signal strength can be increased to a level more conducive to analysis by adding or subtracting signals, resulting in larger signals. The signals from each sensor can be combined by the signal from at least one, some, all or a combination of other sensors to increase the signal strength for higher resolution algorithmic analysis. The combining method can be linear or nonlinear addition, subtraction, multiplication or other transformations.

The controller can be programmed to cancel out external noise that is not associated with the subject laying on the bed. External noise, such as the beat of a bass or the vibrations caused by an air conditioner, register as the same type of signal on all load or other sensors and is therefore canceled out when deltas are combined during processing. Other noise cancellation techniques can be used including, but not limited to, subtraction, combination of the sensor data, adaptive filtering, wavelet transform, independent component analysis, principal component analysis, and/or other linear or nonlinear transforms.

Using superposition analysis, two subjects can be distinguished on one substrate. Superposition simplifies the analysis of the signal with multiple inputs. The usable signal equals the algebraic sum of the responses caused by each independent sensor acting alone. To ascertain the contribution of each individual source, all of the other sources must be calibrated first (turned off or set to zero). This procedure is followed for each source in turn, then the resultant responses are added to determine the true result. The resultant operation is the superposition of the various sources. By using signal strength and out-of-phase heart signal and/or respiration signal, individuals can be distinguished on the same substrate.

The controller can be programmed to provide dynamic center of mass location and movement vectors for the subject, while eliminating those from other subjects and inanimate objects or animals on the substrate. By leveraging multiple sensor assemblies that detect the z-axis of the force vector of gravity, and by discriminating and tracking the center of mass of multiple subjects as they enter and move on a substrate, not only can presence, motion and cardiac and respiratory signals for the subject be determined, but the signals of a single or multiple subjects on the substrate can be enhanced by applying the knowledge of location to the signal received. By analyzing the bio-signal's amplitude and phase in different frequency bands, the center of mass (location) for a subject can be obtained using multiple methods, examples of which include:
DC weight;
AC low band analysis of signal, center of mass (location), respiratory and body position identification of subject;
AC mid band analysis of signal center of mass and cardiac identification of subject; and
AC upper mid band identification of snorer or apnea events.

The data from the load or other sensor assemblies can be used to determine presence and location X and Y, angular orientation, and body positions of a subject on a substrate. Such information is useful for calculating in/out statistics for a subject such as: period of time spent in bed, time when subject fell asleep, time when subject woke up, time spent on back, time spent on side, period of time spent out of bed. The sensor assemblies can be in sleep mode until the presence of a subject is detected on the substrate, waking up the system.

Macro weight measurements can be used to measure the actual static weight of the subject as well as determine changes in weight over time. Weight loss or weight gain can be closely tracked as weight and changes in weight can be measured the entire time a subject is in bed every night. This information may be used to track how different activities or foods affect a person's weight. For example, excessive water retention could be tied to a particular food. In a medical setting, for example, a two-pound weight gain in one night or a five-pound weight gain in one week could raise an alarm that the patient is experiencing congestive heart failure. Unexplained weight loss or weight gain can indicate many medical conditions. The tracking of such unexplained change in weight can alert professionals that something is wrong.

Center of mass can be used to accurately heat and cool particular and limited space in a substrate such as a mattress, with the desired temperature tuned to the specific subject associated with the center of mass, without affecting other subjects on the substrate. Certain mattresses are known to provide heating and/or cooling. As non-limiting examples, a subject can set the controller to actuate the substrate to heat the portion of the substrate under the center of mass when the temperature of the room is below a certain temperature. The subject can set the controller to instruct the substrate to cool the portion of the substrate under the center of mass when the temperature of the room is above a certain temperature.

These macro weight measurements can also be used to determine a movement vector of the subject. Subject motion can be determined and recorded as a trend to determine amount and type of motion during a sleep session. This can determine a general restlessness level as well as other medical conditions such as “restless leg syndrome” or seizures.

Motion detection can also be used to report in real time a subject exiting from the substrate. Predictive bed exit is also possible as the position on the substrate as the subject moves is accurately detected, so movement toward the edge of a substrate is detected in real time. In a hospital or elder care setting, predictive bed exit can be used to prevent falls during bed exit, for example. An alarm might sound so that a staff member can assist the subject exit the substrate safely.

Data from the load or other sensors can be used to detect actual body positions of the subject on the substrate, such as whether the subject is on its back, side, or stomach. Data from the load or other sensors can be used to detect the angular orientation of the subject, whether the subject is aligned on the substrate vertically, horizontally, with his or her head at the foot of the substrate or head of the substrate, or at an angle across the substrate. The sensors can also detect changes in the body positions, or lack thereof. In a medical setting, this can be useful to determine if a subject should be turned to avoid bed sores. In a home or medical setting, firmness of the substrate can be adjusted based on the angular orientation and body position of the subject. For example, body position can be determined from the center of mass, position of heart beat and/or respiration, and directional changes due to blood flow.

Controlling external devices such as lights, ambient temperature, music players, televisions, alarms, coffee makers, door locks and shades can be tied to presence, motion and time, for example. As one example, the controller can collect signals from each load or other sensor, determine if the subject is asleep or awake and control at least one external device based on whether the subject is asleep or awake. The determination of whether a subject is asleep or awake is made based on changes in respiration, heart rate and frequency and/or force of movement. As another example, the controller can collect signals from each load or other sensor, determine that the subject previously on the substrate has exited the substrate and change a status of the at least one external device in response to the determination. As another example, the controller can collect signals from each load sensor, determine that the subject has laid down on the substrate and change a status of the at least one external device in response to the determination.

A light can be automatically dimmed or turned off by instructions from the controller to a controlled lighting device when presence on the substrate is detected. Electronic shades can be automatically closed when presence on the substrate is detected. A light can automatically be turned on when bed exit motion is detected or no presence is detected. A particular light, such as the light on a right side night stand, can be turned on when a subject on the right side of the substrate is detected as exiting the substrate on the right side. Electronic shades can be opened when motion indicating bed exit or no presence is detected. If a subject wants to wake up to natural light, shades can be programmed to open when movement is sensed indicating the subject has woken up. Sleep music can automatically be turned on when presence is detected on the substrate. Predetermined wait times can be programmed into the controller, such that the lights are not turned off or the sleep music is not started for ten minutes after presence is detected, as non-limiting examples.

The controller can be programmed to recognize patterns detected by the load or other sensors. The patterned signals may be in a certain frequency range that falls between the macro and the micro signals. For example, a subject may tap the substrate three times with his or her hand, creating a pattern. This pattern may indicate that the substrate would like the lights turned out. A pattern of four taps may indicate that the subject would like the shades closed, as non-limiting examples. Different patterns may result in different actions. The patterns may be associated with a location on the substrate. For example, three taps near the top right corner of the substrate can turn off lights while three taps near the base of the substrate may result in a portion of the substrate near the feet to be cooled. Patterns can be developed for medical facilities, in which a detected pattern may call a nurse.

While the figures illustrate the use of the load or other sensors with a bed as a substrate, it is contemplated that the load or other sensors can be used with couches, chairs, such as a desk chair, where a subject spends extended periods of time. A wheel chair can be equipped with the sensors to collect signals and provide valuable information about a patient. The sensors may be used in an automobile seat and may help to detect when a driver is falling asleep or his or her leg might go numb. Furthermore, the bed can be a baby's crib, a hospital bed, or any other kind of bed. The substrate can be an air chamber bed, smart scale, smart clothing, electronic clothing, textiles, and the like.

While the figures illustrate the use of the load sensors, other sensors, examples of which are described herein, can be used without departing from the scope of the specification or claims. Other sensors can be vibration sensors, pressure sensors, force sensors, motion sensors and accelerometers as non-limiting examples. In an implementation, the other sensors may be used instead of, in addition to or with the load sensors without departing from the scope of the specification or claims.

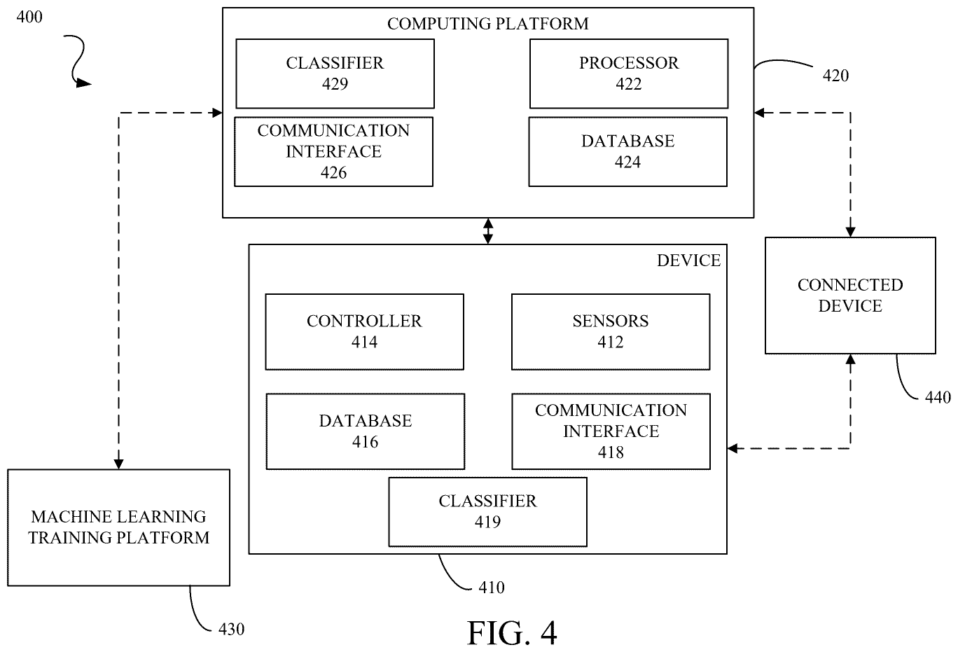

FIG. 4 is a system architecture for a multidimensional multivariate multiple sensor system (MMMSA) 400. The MMMSA 400 includes one or more devices 410 which are connected to or in communication with (collectively “connected to”) a computing platform 420. In an implementation, a machine learning training platform 430 may be connected to the computing platform 420. In an implementation, users may access the data via a connected device 440, which may receive data from the computing platform 420 or the device 410. The connections between the one or more devices 410, the computing platform 420, the machine learning training platform 430, and the connected device 440 can be wired, wireless, optical, combinations thereof and/or the like. The system architecture of the MMMSA 400 is illustrative and may include additional, fewer or different devices, entities and the like which may be similarly or differently architected without departing from the scope of the specification and claims herein. Moreover, the illustrated devices may perform other functions without departing from the scope of the specification and claims herein.

In an implementation, the device 410 can include one or more sensors 412, a controller 414, a database 416, and a communications interface 418. In an implementation, the device 410 can include a classifier 419 for applicable and appropriate machine learning techniques as described herein. The one or more sensors 412 can detect wave patterns of vibration, pressure, force, weight, presence, and motion due to subject(s) activity and/or configuration with respect to the one or more sensors 412. In an implementation, the one or more sensors 412 can generate more than one data stream. In an implementation, the one or sensors 412 can be the same type. In an implementation, the one or more sensors 412 can be time synchronized. In an implementation, the one or more sensors 412 can measure the partial force of gravity on substrate, furniture or other object. In an implementation, the one or more sensors 412 can independently capture multiple external sources of data in one stream (i.e. multivariate signal), for example, weight, heart rate, breathing rate, vibration, and motion from one or more subjects or objects. In an implementation, the data captured by each sensor 412 is correlated with the data captured by at least one, some, all or a combination of the other sensors 412. In an implementation, amplitude changes are correlated. In an implementation, rate and magnitude of changes are correlated. In an implementation, phase and direction of changes are correlated. In an implementation, the one or more sensors 412 placement triangulates the location of center of mass. In an implementation, the one or more sensors 412 can be placed under or built into the legs of a bed, chair, coach, etc. In an implementation, the one or more sensors 412 can be placed under or built into the edges of crib. In an implementation, the one or more sensors 412 can be placed under or built into the floor. In an implementation, the one or more sensors can be placed under or built into a surface area. In an implementation, the one or more sensors 412 locations are used to create a surface map that covers the entire area surrounded by sensors. In an implementation, the one or more sensors 412 can measure data from sources that are anywhere within the area surrounded by the sensors 412, which can be directly on top of the sensor 412, near the sensor 412, or distant from the sensor 412. The one or sensors 416 are not intrusive with respect to the subject(s).

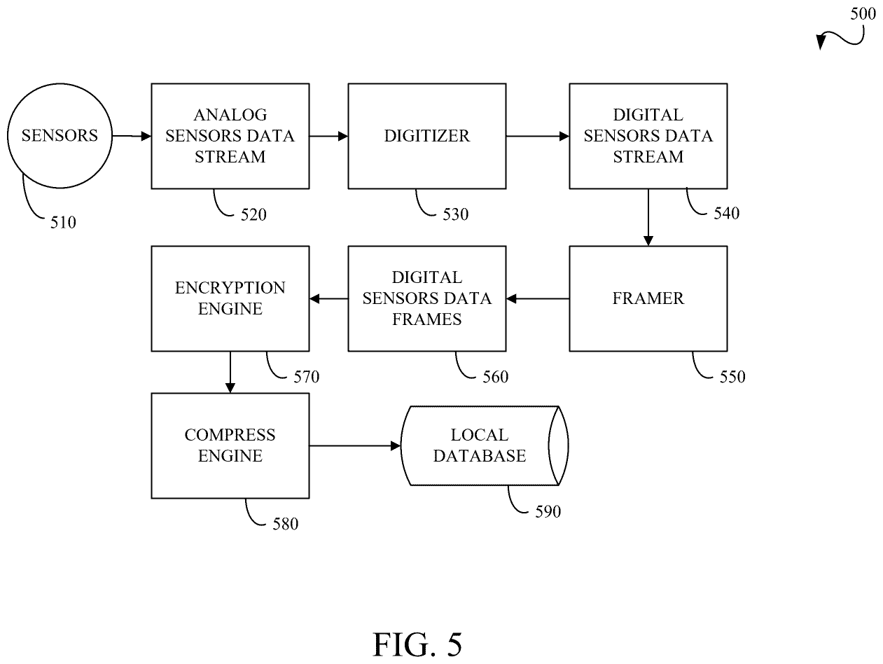

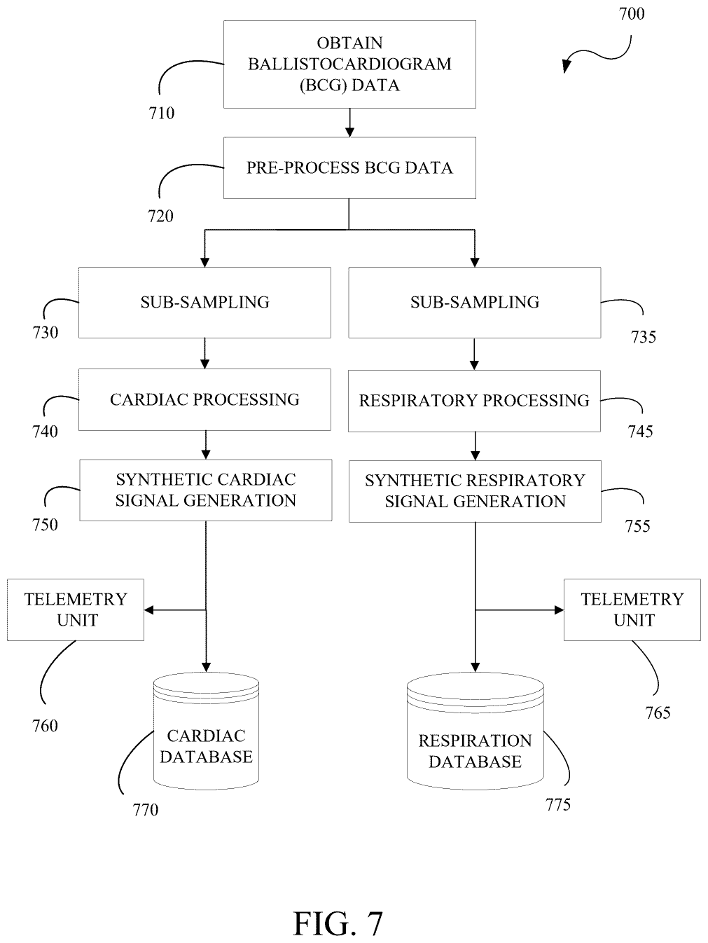

The controller 414 can apply the processes and algorithms described herein with respect to FIGS. 5-15 to the sensor data to determine biometric parameters and other person-specific information for single or multiple subjects at rest and in motion and/or to synthesize cardiac and respiratory signals and associated cardiac and respiratory audio streams. The classifier 419 can apply the processes and algorithms described herein with respect to FIGS. 7, 9, 10, 11, 12, 13 and 15 to the sensor data to determine biometric parameters and other person-specific information for single or multiple subjects at rest and in motion and/or to synthesize cardiac and respiratory signals and associated cardiac and respiratory audio streams. The classifier 419 can apply classifiers to the sensor data to determine the biometric parameters and other person-specific information and/or to synthesize cardiac and respiratory signals and associated cardiac and respiratory audio streams via machine learning. In an implementation, the classifier 419 may be implemented by the controller 414. In an implementation, the sensor data and the biometric parameters and other person-specific information and/or to synthetic cardiac and respiratory signals and associated cardiac and respiratory audio streams can be stored in the database 416. In an implementation, the sensor data, the biometric parameters and other person-specific information, the synthetic cardiac and respiratory signals and associated cardiac and respiratory audio streams, and/or combinations thereof can be transmitted or sent via the communication interface 418 to the computing platform 420 for processing, storage, and/or combinations thereof. The communication interface 418 can be any interface and use any communications protocol to communicate or transfer data between origin and destination endpoints. In an implementation, the device 410 can be any platform or structure which uses the one or more sensors 412 to collect the data from a subject(s) for use by the controller 414 and/or computing platform 420 as described herein. For example, the device 410 may be a combination of the substrate 20, frame 102, legs 104, and multiple load or other sensors 106 as described in FIGS. 1-3. The device 410 and the elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In an implementation, the computing platform 420 can include a processor 422, a database 424, and a communication interface 426. In an implementation, the computing platform 420 may include a classifier 429 for applicable and appropriate machine learning techniques as described herein. The processor 422 can obtain the sensor data from the sensors 412 or the controller 414 and can apply the processes and algorithms described herein with respect to FIGS. 5-15 to the sensor data to determine biometric parameters and other person-specific information for single or multiple subjects at rest and in motion and/or to synthesize cardiac and respiratory signals and associated cardiac and respiratory audio streams. In an implementation, the processor 422 can obtain the biometric parameters and other person-specific information and/or the synthetic cardiac and respiratory signals and associated cardiac and respiratory audio streams from the controller 414 to store in database 424 for temporal and other types of analysis. In an implementation, the classifier 429 can apply the processes and algorithms described herein with respect to FIGS. 5-15 to the sensor data to determine biometric parameters and other person-specific information for single or multiple subjects at rest and in motion and/or to synthesize cardiac and respiratory signals and associated cardiac and respiratory audio streams. The classifier 429 can apply classifiers to the sensor data to determine the biometric parameters and other person-specific information and/or to synthesize cardiac and respiratory signals and associated cardiac and respiratory audio streams via machine learning. In an implementation, the classifier 429 may be implemented by the processor 422. In an implementation, the sensor data and the biometric parameters and other person-specific information and/or synthetic cardiac and respiratory signals and associated cardiac and respiratory audio streams can be stored in the database 424. The communication interface 426 can be any interface and use any communications protocol to communicate or transfer data between origin and destination endpoints. In an implementation, the computing platform 420 may be a cloud-based platform. In an implementation, the processor 422 can be the cloud-based computer 212 or off-site controller 214. The computing platform 420 and elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In an implementation, the machine learning training platform 430 can access and process sensor data to train and generate classifiers. The classifiers can be transmitted or sent to the classifier 429 or to the classifier 419.

FIG. 5 is a processing pipeline 500 for obtaining sensor data such as, but not limited to, load sensor data and other sensor data. An analog sensors data stream 520 is received from the sensors 510. A digitizer 530 digitizes the analog sensors data stream into a digital sensors data stream 540. A framer 550 generates digital sensors data frames 560 from the digital sensors data stream 540 which includes all the digital sensors data stream values within a fixed or adaptive time window. An encryption engine 570 encodes the digital sensors data frames 560 such that the data is protected from unauthorized access. A compression engine 580 compresses the encrypted data to reduce the size of the data that is going to be saved in the database 590. This reduces cost and provides faster access during read time. The processing pipeline 500 shown in FIG. 5 is illustrative and can include any, all, none or a combination of the blocks or modules shown in FIG. 5. The processing order shown in FIG. 5 is illustrative and the processing order may vary without departing from the scope of the specification or claims.

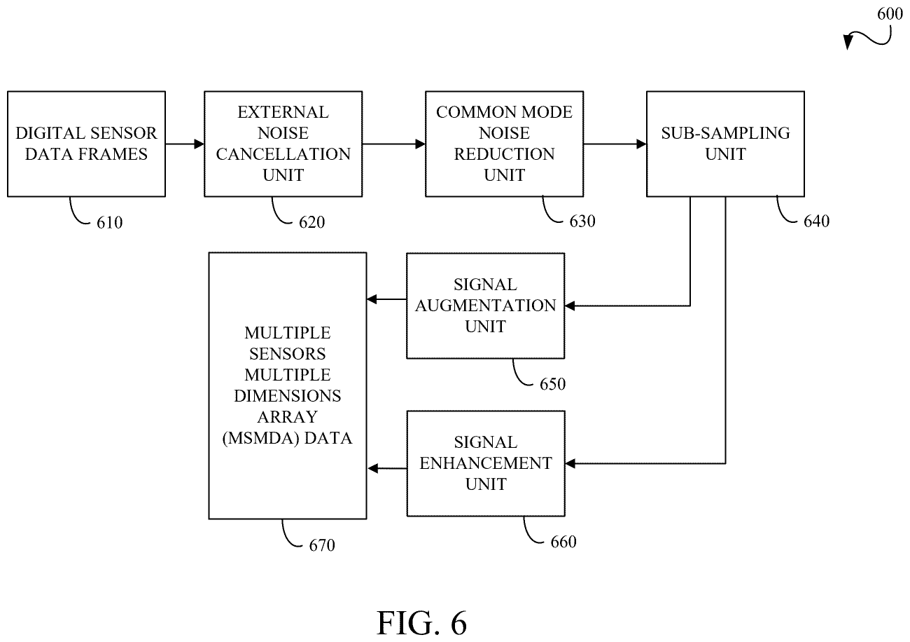

FIG. 6 is a pre-processing pipeline 600 for processing the sensor data into multiple sensors multiple dimensions array (MSMDA) data. The pre-processing pipeline 600 shown in FIG. 6 is illustrative and can include any, all, none or a combination of the blocks or modules shown in FIG. 6. The processing order shown in FIG. 6 is illustrative and the processing order may vary without departing from the scope of the specification or claims. The pre-processing pipeline 600 processes digital sensor data frames 610. An external noise cancellation unit 620 removes or attenuates noise sources that might have the same or different level of impact on each sensor. The external noise cancellation unit 620 can use a variety of techniques including, but not limited to, subtraction, combination of the input data frames, adaptive filtering, wavelet transform, independent component analysis, principal component analysis, and/or other linear or nonlinear transforms. A common mode noise reduction unit 630 removes or attenuates noises which are captured equally by all sensors. The common mode noise reduction unit 630 may use a variety of techniques including, but not limited to, subtraction, combination of the input data frames, adaptive filtering, wavelet transform, independent component analysis, principal component analysis, and/or other linear or nonlinear transforms. A subsampling unit 640 samples the digital sensor data and can include downsampling, upsampling or resampling. The subsampling unit 640 can be implemented as a multi-stage sampling or multi-phase sampling. A signal augmentation unit 650 can improve the energy of the data or content. The signal augmentation unit 650 can be implemented as scaling, normalization, log transformation, power transformation, linear or nonlinear combination of input data frames and/or other transformations on the input data frames. A signal enhancement unit 660 can improve the signal to noise ratio of the input data. The signal enhancement unit 660 can be implemented as a linear or nonlinear combination of input data frames. For example, the signal enhancement unit 660 may combine the signal deltas to increase the signal strength for higher resolution algorithmic analysis. The pre-processing pipeline 600 outputs MSMDA data 670, which is the primary input to the methods described herein.

FIG. 7 is a flowchart of a method 700 for generating synthetic cardio-respiratory signals. The processing order shown in FIG. 7 is illustrative and the processing order may vary without departing from the scope of the specification or claims. The method 700 includes: obtaining 710 ballistocardiogram (BCG) data; pre-processing 720 the BCG data; sub-sampling 730 the pre-processed cardiac BCG data for cardiac signal synthetization and sub-sampling 735 the pre-processed respiratory BCG data for respiratory signal synthetization; cardiac processing 740 the sub-sampled cardiac BCG data and respiratory processing 745 the sub-sampled respiratory BCG data; generating 750 synthetic cardiac signals and generating 755 synthetic respiratory signals; transmitting 760 the synthetic cardiac signals to a telemetry unit and transmitting 760 the synthetic respiratory signals to a telemetry unit; and storing 770 the synthetic cardiac signals in a database and storing 775 the synthetic respiratory signals in a database.

The method 700 includes obtaining 710 ballistocardiogram (BCG) data. The BCG data can be obtained from one or more sensors implemented in a substrate as described herein.

The method 700 includes pre-processing 720 the BCG data. The BCG data is pre-processed using one or more signal processing techniques known or to be known. The pre-processing techniques can include any signal processing techniques with assist in the separation of cardiac associated signals and respiration associated signals from the BCG data. These techniques can include filtering, artifact removal, noise reduction, signal enhancement, augmentation, normalization, standardization, resampling, and combinations thereof.

The method 700 includes sub-sampling 730 the pre-processed cardiac BCG data for cardiac signal synthetization and sub-sampling 735 the pre-processed respiratory BCG data for respiratory signal synthetization. Each of the pre-processed cardiac BCG data and the pre-processed respiratory BCG data are sub-sampled to change the sampling rate to sampling rates which are optimal for cardiac signal synthetization or respiratory signal synthetization, respectively. For example, each of a cardiac sampling rate and a respiratory sampling rate depends on the type of processing used to generate the synthetic cardiac signal or synthetic respiratory signal as described herein, the morphology of the pre-processed cardiac BCG data and the pre-processed respiratory BCG data as described herein, the cardiac components and respiratory components as described herein, and the associated component to noise ratios. The sub-sampling techniques can include downsampling, upsampling or resampling. The subsampling can be implemented as a multi-stage sampling or multi-phase sampling.

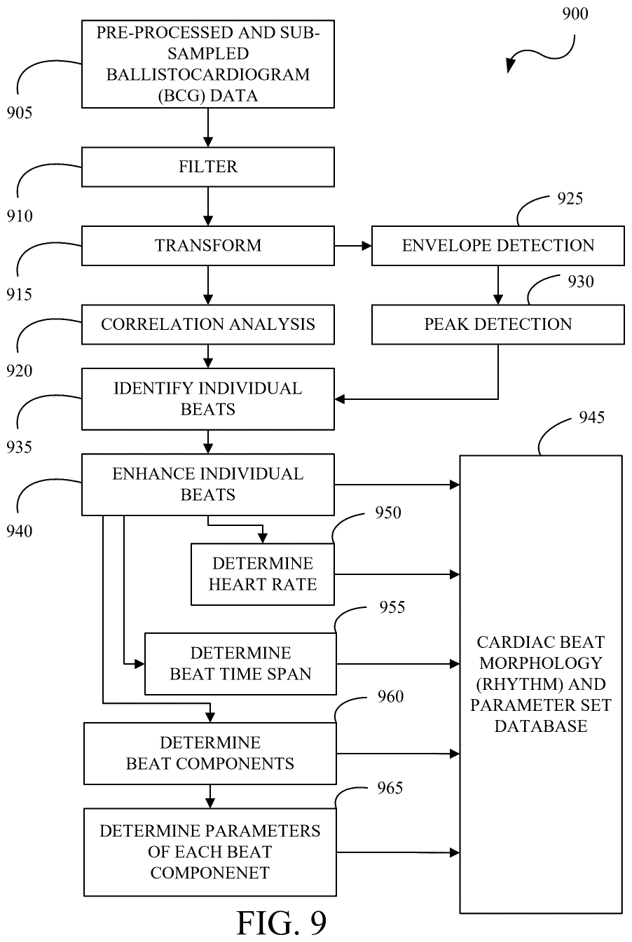

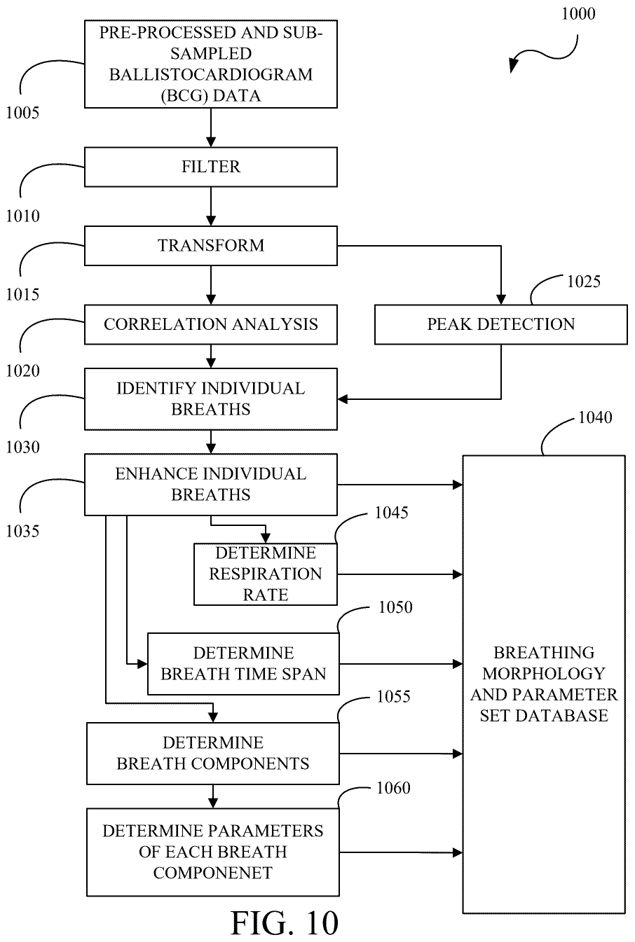

The method 700 includes cardiac processing 740 the sub-sampled cardiac BCG data and respiratory processing 745 the sub-sampled respiratory BCG data. The sub-sampled cardiac BCG data is processed as described herein with respect to FIG. 9 and FIGS. 11-15 as applicable and appropriate. The sub-sampled respiratory BCG data is processed as described herein with respect to FIG. 10 and FIGS. 11-15 as applicable and appropriate.

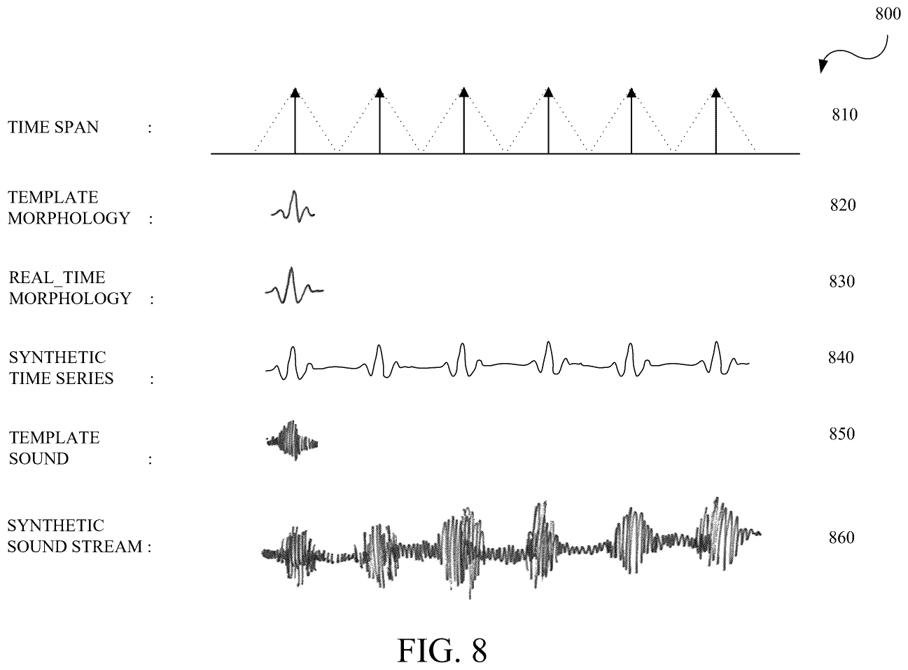

The method 700 includes generating 750 synthetic cardiac signals and generating 755 synthetic respiratory signals. The cardiac processed BCG data is processed as described herein with respect to FIG. 9 and FIGS. 11-15 as applicable and appropriate to generate synthetic cardiac signals which include a cardiac time series and a cardiac sound stream as shown in FIG. 8, for example. The respiratory processed BCG data is processed as described herein with respect to FIG. 10 and FIGS. 11-15 as applicable and appropriate to generate synthetic respiratory signals which include a respiratory time series and a respiratory sound stream as shown in FIG. 8, for example.

The method 700 includes transmitting 760 the synthetic cardiac signals to a telemetry unit and transmitting 760 the synthetic respiratory signals to a telemetry unit. Each of the generated synthetic cardiac signals and generated synthetic respiratory signals can be sent to a telemetry unit, which in turn can make the generated synthetic cardiac signals and generated synthetic respiratory signals available to remote users such as, for example, a physician or remote care giver.

The method 700 includes storing 770 the synthetic cardiac signals in a database and storing 775 the synthetic respiratory signals in a database. Each of the generated synthetic cardiac signals and generated synthetic respiratory signals can be stored in a local or remote database. In an implementation, one or more databases can be used to store the generated synthetic cardiac signals and generated synthetic respiratory signals.

FIG. 8 is an example of signals 800 generated in the method of generating synthetic cardio-respiratory signals. As described herein, the cardiac processing and the respiratory processing generates a number of components which are used to synthesize synthetic cardiac signals and synthetic respiratory signals (collectively “cardio-respiratory signals”), respectively. As described herein for the process used for obtaining the components, some of the components include a time span 810 which is determined between dominant components in pre-synthetic cardio-respiratory signals, a template morphology 820 which is obtained from one or more databases, a real-time morphology 830 based on the template morphology 820 and other parameters described herein, a synthetic time series 840 based on the time span 810 and the real-time morphology 830, a template sound 850 which is obtained from one or more databases or from the real-time morphology 830, and a synthetic sound stream 860 based on the time span 810 and the template sound 850.

FIG. 9 is a flowchart of a method 900 for generating synthetic cardio signals. The method 900 includes: pre-processing and sub-sampling 905 BCG data; filtering 910 the pre-processed and sub-sampled cardiac BCG data; transforming 915 the filtered cardiac BCG data; performing 920 correlation analysis on the transformed cardiac BCG data; performing 925 envelope detection on the transformed cardiac BCG data; performing 930 peak detection on the envelope detected cardiac BCG data; identifying 935 individual cardiac beats based on the correlated cardiac BCG data and the peak detected cardiac BCG data; enhancing 940 the individual cardiac beats; storing 945 the individual cardiac beats in a cardiac beat morphology (rhythm) and parameter set database or cardiac database; determining 950 heart rate from the individual cardiac beats and storing 945 same; determining 955 time span from the individual cardiac beats and storing 945 same; determining 960 beat components from the individual cardiac beats and storing 945 same; and determining 965 beat parameters of each beat component and storing 945 same. Cardiac beats are an illustrative cardiac event and other cardiac events can be determined such as heart beat pattern change rate, and heart beat with normal pattern and heart beat with abnormal pattern.

The method 900 includes pre-processing and sub-sampling 905 BCG data. The BCG data is processed as described herein and as described with respect to the pre-processing 720 and the sub-sampling 730 of FIG. 7.

The method 900 includes filtering 910 the pre-processed and sub-sampled cardiac BCG data. The filtering 910 is designed to eliminate or remove components of the pre-processed and sub-sampled cardiac BCG data which do not pertain to cardiac processing. That is, the filtering 910 retains those components which are representative of the cardiac information. In illustrative examples, the filtering 910 can preserve the diastolic and systolic components of the pre-processed and sub-sampled BCG data 905, the atrial and ventricular components of the pre-processed and sub-sampled BCG data 905, the cardiac beat waveforms or heartbeat oscillations in the pre-processed and sub-sampled BCG data 905, and the spectral components within the cardiac frequency band in the pre-processed and sub-sampled BCG data 905. In illustrative examples, the filtering 910 can remove the breathing related components of the pre-processed and sub-sampled BCG data 905 and other components outside the cardiac frequency band in the pre-processed and sub-sampled BCG data 905. The filtering 910 can use infinite impulse response (IIR) filter processing, finite impulse response (FIR) filter processing, or combinations thereof. The filtering 910 can use low pass filters, high pass filters, bandpass filters, bandstop filters, notch filters, or combinations thereof.

The method 900 includes transforming 915 the filtered cardiac BCG data. The transforming 915 enhances the cardiac components by modeling the filtered cardiac BCG data as a collection of waveforms of a particular form that resemble the cardiac morphology, where each waveform type is associated with one or more transforms. For example, but not limited to, the collection of waveforms can be sinusoids, mother wavelets, periodic basis functions, and the like, and an associated transform processes can be Fourier transforms, wavelet transforms, and periodicity transforms. The transforming 915 can also use cosine transforms or mathematical transform operations such as root-mean-square, absolute, moving average, moving median, and the like. The transforming 915 is used to enhance those components which are representative of the cardiac information.

The method 900 includes performing 920 correlation analysis on the transformed cardiac BCG data. The performing 920 can use correlation techniques to measure the strength of relationships between different segments of the transformed cardiac BCG data. For example, but not limited to, the correlation techniques can include linear and nonlinear methods. Correlation analysis is used in later processing to determine identify each beat or beat locations.

The method 900 includes performing 925 envelope detection on the transformed cardiac BCG data. Envelop detection is performed on a relatively high-frequency amplitude modulated signal (input signal) of the transformed cardiac BCG data and provides an output which is equivalent to an outline of the input signal as described by connecting all the local peaks in the input signal. For example, but not limited to, envelope detection can use a low pass filter, a Hilbert transform, or other envelope detection methods. Envelope detection is used to help determine start and stop points of a beat.

The method 900 includes performing 930 peak detection on the envelope detected cardiac BCG data. Peak detection is performed to find local maximum and minimum points of the envelope detected cardiac BCG data. For example, peak detection can return all peaks, all valleys, dominant peaks, dominant valleys, or combinations thereof. Peak detection is used to help determine a center of the peak, which in turn is used later to determine the cardiac morphology including, but not limited to, the number of beats, time span, frequency, and width.

The method 900 includes identifying 935 individual cardiac beats based on the correlated cardiac BCG data and the peak detected cardiac BCG data. The information available from the correlation analysis and peak detection is collectively used to identify individual cardiac beats.

The method 900 includes enhancing 940 the individual cardiac beats. The identified individual beats undergo signal enhancement by applying a window, a factor, a transform, or like techniques to enhance specific characteristics of the individual cardiac beats which are representative of the cardiac information. The cardiac beats are indicative of a cardiac beat morphology.

The method 900 includes storing 945 the individual cardiac beats in a cardiac beat morphology (rhythm) and parameter set database. The results from signal enhancement are stored in the cardiac beat morphology (rhythm) and parameter set database. The cardiac beat morphology (rhythm) and parameter set database can be one or more databases. For example, the cardiac beat morphology (rhythm) and parameter set database can include a normal or baseline database and one or more abnormal or disease databases. The normal or baseline database can include a cardiac beat morphology (rhythm) and parameter set which is established or identified as a baseline cardiac beat morphology (rhythm) and parameter set against which later cardiac beat morphology (rhythm) and parameter sets can be compared to determine any variances.

The method 900 includes determining 950 heart rate from the enhanced individual cardiac beats and storing 945 the same. Heart rate information is determined from the enhanced individual cardiac beats by using time domain, frequency domain, time frequency domain analysis, or combinations thereof. The heart rate information is stored in the cardiac beat morphology (rhythm) and parameter set database.

The method 900 includes determining 955 time span from the enhanced individual cardiac beats and storing 945 same. Dominant components, onset points, and offset points are determined from the enhanced individual cardiac beats. The dominant components can be, for example, the center of the cardiac beat or the peak of the cardiac beat. The onset and offset points are collectively used to define or determine a time span as shown in FIG. 8. The dominant components, onset points, offset points, and time span are stored in the cardiac beat morphology (rhythm) and parameter set database.

The method 900 includes determining 960 beat components from the enhanced individual cardiac beats and storing 945 same. Beat components are determined for each of the enhanced individual cardiac beats. The beat components depend on the cardiac model. For example, the beat components can be P, Q, R, S and T waveforms, diastolic/systolic waveforms, or atrial/ventricular depolarization and repolarization. These are illustrative and other beat components can be used. The beat components are stored in the cardiac beat morphology (rhythm) and parameter set database.

The method 900 includes determining 965 beat parameters of each beat component and storing 945 same. Parameters are determined for each of the beat components. These parameters can include, but are not limited to, amplitude, location, onset, offset, peak, width, duration, slope, latency or other parameters. The parameters for each of the beat components are stored in the cardiac beat morphology (rhythm) and parameter set database. The individual beats morphology (rhythm), heart rate, time span, beat components, and parameters collectively constitute the cardiac parameter set.

FIG. 10 is a flowchart of a method 1000 for generating synthetic respiratory signals. The method 1000 includes: pre-processing and sub-sampling 1005 BCG data; filtering 1010 the pre-processed and sub-sampled respiratory BCG data; transforming 1015 the filtered respiratory BCG data; performing 1020 correlation analysis on the transformed respiratory BCG data; performing 1025 peak detection on the on the transformed respiratory BCG data; identifying 1030 individual breaths based on the correlated respiratory BCG data and the peak detected respiratory BCG data; enhancing 1035 the individual breaths; storing 1040 the individual breaths in a breathing morphology and parameter set database or respiratory database; determining 1045 respiration rate from the individual breaths and storing 1040 the same; determining 1050 breath time span from the individual breaths and storing 1040 the same; determining 1055 breath components from the individual breaths and storing 1040 the same; and determining 1060 parameters of each breath component and storing 1040 the same. Breaths are an illustrative respiratory event and other respiratory events can be determined such as snores and associated data such as snoring rate, snore during inhalation, and snore during exhalation, or breath pattern changes and associated data such as a breath pattern change rate, and breathing with normal pattern and breathing with abnormal pattern.

The method 1000 includes pre-processing and sub-sampling 1005 BCG data. The BCG data is processed as described herein and as described with respect to the pre-processing 720 and the sub-sampling 735 of FIG. 7.

The method 1000 includes filtering 1010 the pre-processed and sub-sampled respiratory BCG data. The filtering 1010 is designed to eliminate or remove components of the pre-processed and sub-sampled respiratory BCG data which do not pertain to respiratory processing. That is, the filtering 1010 retains those components which are representative of the respiratory information. In illustrative examples, the filtering 1010 can preserve the inspiration (inhalation) and expiration (exhalation) components of the pre-processed and sub-sampled BCG data 1005, the snore or the breathing sound vibrations present in the pre-processed and sub-sampled BCG data 1005, or the spectral components within the respiratory frequency band in the pre-processed and sub-sampled BCG data 1005. In illustrative examples, the filtering 1010 can remove the cardiac related components of the pre-processed and sub-sampled BCG data 1005 and/or other components outside the respiration frequency band in the pre-processed and sub-sampled BCG data 1005. The filtering1010 can use infinite impulse response (IIR) filter processing, finite impulse response (FIR) filter processing, or combinations thereof. The filtering 1010 can use low pass filters, high pass filters, bandpass filters, bandstop filters, notch filters, or combinations thereof.

The method 1000 includes transforming 1015 the filtered respiratory BCG data. The transforming 1015 enhances the respiratory components by modeling the filtered respiratory BCG data as a collection of waveforms of a particular form that resemble the respiratory morphology, where each waveform type is associated with one or more transforms. For example, but not limited to, the collection of waveforms can be sinusoids, mother wavelets, periodic basis functions, and the like, and associated transform processes can be Fourier transforms, wavelet transforms, and periodicity transforms. The transforming 1015 can also use cosine transforms or mathematical transform operations such as root-mean-square, absolute, moving average, moving median, and the like. The transforming 1015 is used to enhance those components which are representative of the respiratory information.

The method 1000 includes performing 1020 correlation analysis on the transformed respiratory BCG data. The performing 1020 can use correlation techniques to measure the strength of relationships between different segments of the transformed respiratory BCG data. For example, but not limited to, the correlation techniques can include linear and nonlinear methods. Correlation analysis is used in later processing to determine identify each breath.

The method 1000 includes performing 1025 peak detection on the on the transformed respiratory BCG data. Peak detection is performed to find local maximum and minimum points of the transformed respiratory BCG data. For example, peak detection can return all peaks, all valleys, dominant peaks, dominant valleys, or combinations thereof. Peak detection is used to help determine a center of the peak, which in turn is used later to determine the transformed respiratory BCG data morphology.

The method 1000 includes identifying 1030 individual breaths based on the correlated respiratory BCG data and the peak detected respiratory BCG data. The information available from the correlation analysis and peak detection is collectively used to identify individual breaths. The breaths are indicative of a breathing morphology.

The method 1000 includes enhancing 1035 the individual breaths. The identified individual breaths undergo signal enhancement by applying a window, a factor, a transform, or like techniques to enhance specific characteristics of the individual breaths which are representative of the respiratory information.

The method 1000 includes storing 1040 the individual breaths in a breathing morphology and parameter set database. The results from signal enhancement are stored in the breathing morphology and parameter set database. The breathing morphology and parameter set database can be one or more databases. For example, the breathing morphology and parameter set database can include a normal or baseline database and one or more abnormal or disease databases. The normal or baseline database can include a breathing morphology and parameter set which is established or identified as a baseline breathing morphology and parameter set against which later breathing morphology and parameter sets can be compared to determine any variances.

The method 1000 includes determining 1045 respiration rate from the individual breaths and storing 1040 the same. Respiration rate information is determined from the enhanced individual breaths by using time domain, frequency domain, time frequency domain analysis, or combinations thereof. The respiration rate information is stored in the breathing morphology and parameter set database.

The method 1000 includes determining 1050 breath time span from the individual breaths and storing 1040 the same. Dominant components, onset points, and offset points are determined from the enhanced individual breaths. The dominant components can be, for example, the center of the breath or the peak of the breath. The onset and offset points are collectively used to define or determine a time span as shown in FIG. 8. The dominant components, onset points, offset points, and time span are stored in the breathing morphology and parameter set database.

The method 1000 includes determining 1055 breath components from the individual breaths and storing 1040 the same. Breath components are determined for each of the enhanced individual breaths. The breath components can be, for example, inhale, exhale, inspiration, expiration, and the like. These are illustrative and other breath components can be used. The breath components are stored in the breathing morphology and parameter set database.

The method 1000 includes determining 1060 parameters of each breath component and storing 1040 the same. Parameters are determined for each of the breath components. These parameters can include, but are not limited to, amplitude, location, onset, offset, peak, width, duration, slope, latency or other parameters. The parameters for each of the breath components are stored in the breathing morphology and parameter set database. The individual breaths morphology (rhythm), respiration rate, time span, breath components, and parameters collectively constitute the respiratory parameter set.

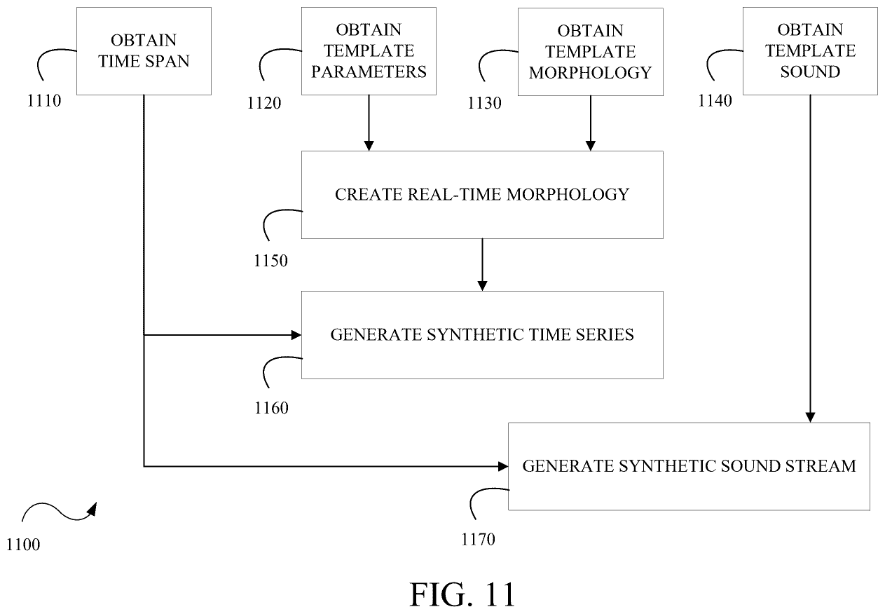

FIG. 11 is a flowchart of a method 1100 for generating synthetic cardio-respiratory time series and sound streams using a defined template sound. The method 1100 includes: obtaining 1110 time span information from a cardiac (or respiratory) database; obtaining 1120 template parameters from a cardiac (or respiratory) database; obtaining 1130 template morphology from a cardiac (or respiratory) database; obtaining 1140 template sound from a cardiac (or respiratory) sound database; creating 1150 a real-time morphology from the template parameters and the template morphology; generating 1160 a synthetic time series from the time span information and the real-time morphology; and generating 1170 a synthetic sound stream for the synthetic time series based on the time span and the template sound.

The method 1100 includes obtaining 1110 time span information from a cardiac (or respiratory) database. The cardiac and respiratory time span information are obtained from a cardiac database as described in FIG. 9 and from a respiratory database as described in FIG. 10, respectively.

The method 1100 includes obtaining 1120 template parameters from a cardiac (or respiratory) database. The cardiac and respiratory template parameters are obtained from a cardiac database as described in FIG. 9 and from a respiratory database as described in FIG. 10, respectively.

The method 1100 includes obtaining 1130 template morphology from a cardiac (or respiratory) database. The beat or breath template morphology is obtained from a cardiac database as described in FIG. 9 or from a respiratory database as described in FIG. 10, respectively. The respective databases can contain one or more template morphologies for different conditions, diseases, ailments, and the like. In an implementation, the template morphology can be obtained from a standalone or separate database or dictionary of pre-recorded cardiac beats or respiratory breaths. In an implementation, the template morphology can be obtained by using functions to transform a BCG morphology into a cardiac, such as an electrocardiogram (ECG) morphology or a breath morphology. For example, an inverse mapping can be used to generate characteristic points or waves for an ECG (for example, P, Q, R, S and T) from the characteristic points or waves of the BCG (H, I, J, K, L, M, N). For example, mathematical or statistical models (such as a Gaussian mixture model) can be used to create simulated cardiac beats or respiratory breaths. The model can use default parameters or the template parameters to simulate the cardiac beats or respiratory breaths.

The method 1100 includes obtaining 1140 template sound from a database. A database has one or more template sounds which can represent normal condition, multiple cardiac conditions, multiple respiratory conditions, multiple cardio-respiratory conditions, and the like.

The method 1100 includes creating 1150 a real-time morphology from the template parameters and the template morphology. Mathematical transformations, windowing functions, or like transforms or functions can be used to adjust or convert the template morphology into a real-time morphology using the template parameters. For example, the template morphology can be stretched, compressed, realigned, and the like in relation to the template parameters. A real-time morphology is shown in FIG. 8.

The method 1100 includes generating 1160 a synthetic time series from the time span information and the real-time morphology. The real-time morphology is effectively reproduced based on the time span. In an implementation, a convolution function can be used to convolve the real-time morphology with an impulse train located at the center of time spans. The synthetic time series is shown in FIG. 8.

The method 1100 includes generating 1170 a synthetic sound stream for the synthetic time series based on the time span and the template sound. The template sound is effectively reproduced based on the time span. In an implementation, a convolution function can be used to convolve the template sound with an impulse train located at the center of time spans. In an implementation, a modulation function such as amplitude modulation, frequency modulation, phase modulation or a combination thereof can be applied to the template sound which is in sync with the time span. The synthetic sound stream and the synthetic time series are run in synchronization to simulate an audible beating heart or pumping respiratory function, for example.

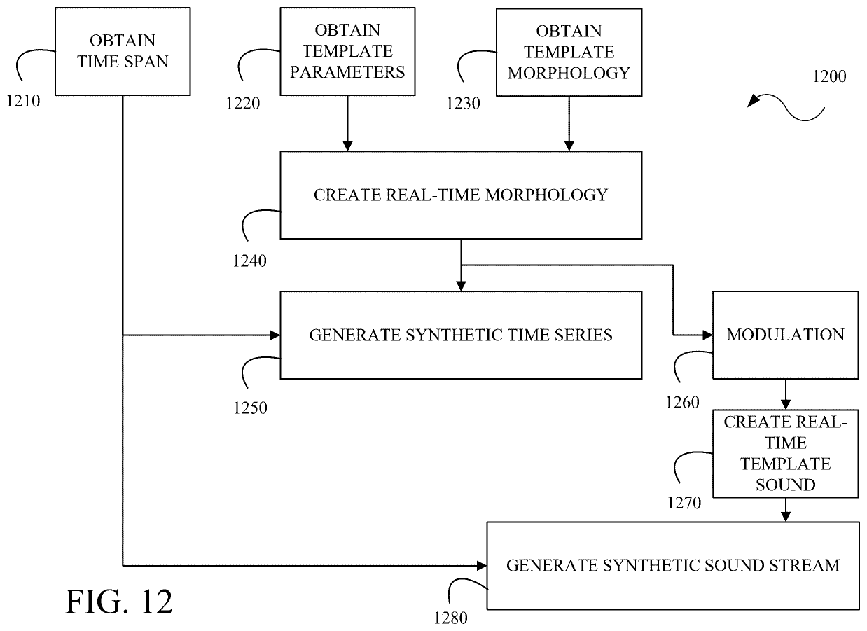

FIG. 12 is a flowchart of a method 1200 for generating synthetic cardio-respiratory time series and sound streams using an adaptive template sound. The method 1200 includes: obtaining 1210 time span information from a cardiac (or respiratory) database; obtaining 1220 template parameters from a cardiac (or respiratory) database; obtaining 1230 template morphology from a cardiac (or respiratory) database; creating 1240 a real-time morphology from the template parameters and the template morphology; generating 1250 a synthetic time series from the time span information and the real-time morphology; modulating 1260 the real-time morphology; creating 1270 a real-time template sound from the modulated real-time morphology; and generating 1280 a synthetic sound stream for the synthetic time series based on the time span and the real-time template sound.

The method 1200 includes obtaining 1210 time span information from a cardiac (or respiratory) database. The cardiac and respiratory time span information are obtained from a cardiac database as described in FIG. 9 and from a respiratory database as described in FIG. 10, respectively.

The method 1200 includes obtaining 1220 template parameters from a cardiac (or respiratory) database. The cardiac and respiratory template parameters are obtained from a cardiac database as described in FIG. 9 and from a respiratory database as described in FIG. 10, respectively.

The method 1200 includes obtaining 1230 template morphology from a cardiac (or respiratory) database. The beat or breath template morphology is obtained from a cardiac database as described in FIG. 9 or from a respiratory database as described in FIG. 10, respectively. The respective databases can contain one or more template morphologies for different conditions, diseases, ailments, and the like. In an implementation, the template morphology can be obtained from a standalone or separate database or dictionary of pre-recorded cardiac beats or respiratory breaths. In an implementation, the template morphology can be obtained by using functions to transform a BCG morphology into a cardiac, such as an electrocardiogram (ECG) morphology or a breath morphology. For example, an inverse mapping can be used to generate characteristic points or waves for an ECG (for example, P, Q, R, S and T) from the characteristic points or waves of the BCG (H, I, J, K, L, M, N). For example, mathematical or statistical models (such as a Gaussian mixture model) can be used to create simulated cardiac beats or respiratory breaths. The model can use default parameters or the template parameters to simulate the cardiac beats or respiratory breaths.

The method 1200 includes creating 1240 a real-time morphology from the template parameters and the template morphology. Mathematical transformations, windowing functions, or like transforms or functions can be used to adjust or convert the template morphology into a real-time morphology using the template parameters. For example, the template morphology can be stretched, compressed, realigned, and the like in relation to the template parameters. A real-time morphology is shown in FIG. 8.

The method 1200 includes generating 1250 a synthetic time series from the time span information and the real-time morphology. The real-time morphology is effectively reproduced based on the time span. In an implementation, a convolution function can be used to convolve the real-time morphology with an impulse train located at the center of time spans. The synthetic time series is shown in FIG. 8.

The method 1200 includes modulating 1260 the real-time morphology. In an implementation, the real-time morphology is modulated using for example, but not limited to, amplitude modulation, frequency modulation, phase modulation, or a combination thereof to achieve human audio frequency range. In an implementation, one or more modulation techniques can be used to vary one or more properties of the real-time morphology waveform to achieve human audio frequency range.

The method 1200 includes creating 1270 a real-time template sound from the modulated real-time morphology.

The method 1200 includes generating 1280 a synthetic sound stream for the synthetic time series based on the time span and the real-time template sound. The real-time template sound is effectively reproduced based on the time span. In an implementation, a convolution function can be used to convolve the real-time template sound with an impulse train located at the center of time spans. In an implementation, a modulation function such as amplitude modulation, frequency modulation, phase modulation or a combination thereof can be applied to the real-time template sound which is in sync with the time span. The synthetic sound stream and the synthetic time series are run in synchronization to simulate an audible beating heart or pumping respiratory function, for example.

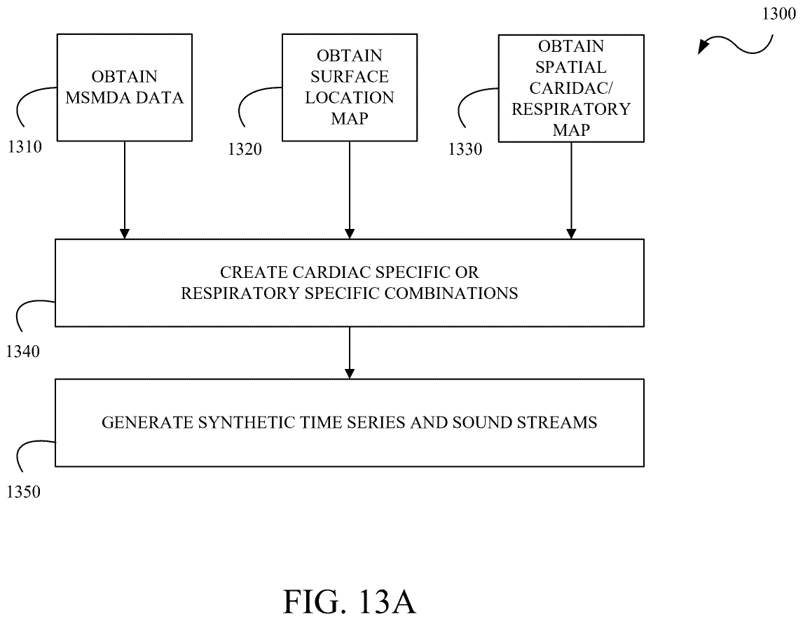

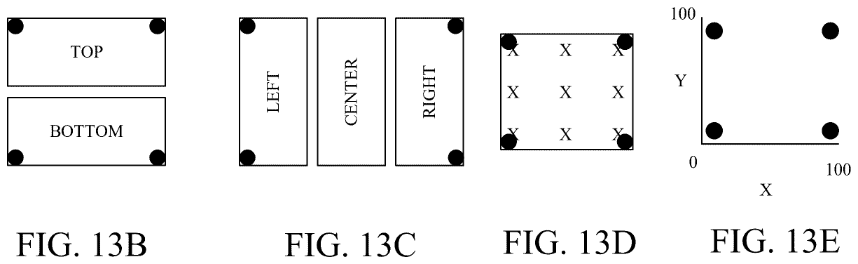

FIG. 13 is a flowchart of a method 1300 for generating synthetic cardio-respiratory signals from a multiple sensor multiple dimensions system. The method 1300 includes obtaining 1310 multiple sensor multiple dimensions array (MSMDA) data; obtaining 1320 surface location map of one or more sensors; obtaining 1330 spatial cardiac or respiratory map(s); creating 1340 cardiac specific or respiratory specific combinations; and generating 1350 synthetic time series and sound streams.

The method 1300 includes obtaining 1310 multiple sensor multiple dimensions array (MSMDA) data. In the event that multiple BCG sensors are available, MSMDA data is obtained using the pre-processing pipeline 600 for processing the sensor data into MSMDA data as described with respect to FIG. 6, for example.

The method 1300 includes obtaining 1320 surface location map of one or more sensors. A two-dimensional surface location map is generated to represent the surface of a substrate, furniture or other object. FIGS. 13A-D show example surface location maps for a multidimensional multivariate multiple sensors system with 4 sensors. FIG. 13A shows mapping the surface into a top section and bottom section. FIG. 13B shows mapping the surface into left, center, and right sections. FIG. 13C shows mapping the surface into 9 coordinates: top left, middle top, top right, middle right, bottom right, middle bottom, bottom left, middle left, and center. FIG. 13D shows mapping the surface into a two dimensional X-Y coordinate, where X and Y are in the range of 0-100 such that (X, Y)=(0,0) represents the bottom left corner of the surface, (X, Y)=(100,100) represent the top right corner of the surface, and (X, Y)=(50,50) shows the center of the surface. The coordinate system is illustrative and other formats can be used. The surface location maps are illustrative and other formats can be used.

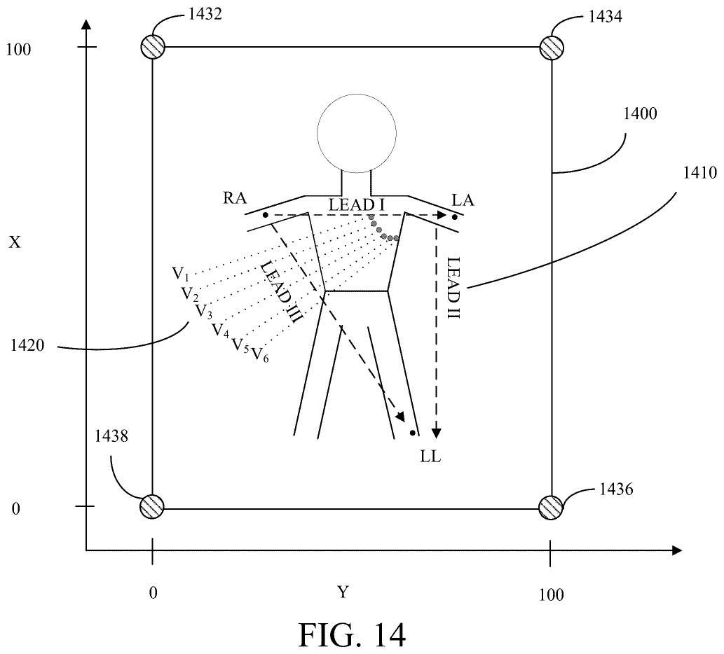

The method 1300 includes obtaining 1330 spatial cardiac or respiratory map(s). The spatial cardiac or respiratory map(s) can be standard electrocardiogram respiratory maps or can be customized maps that model the body surface potential to different views of the heart and lungs. Examples include unipolar ECG configuration, bipolar ECG configuration, pericardial ECG configuration, chest and abdominal respiratory configuration, inferior or superior configuration, anterior or posterior configuration, and like configurations. FIG. 14 is an example of a surface location map 1400 and spatial cardio-respiratory maps 1410 and 1420. The surface location map 1400 illustrates the location of multiple sensors 1432, 1434, 1436 and 1438 in a two dimensional X-Y coordinate, where X and Y are in the range of 0-100 such that (X, Y)=(0,0) represents the bottom left corner of the surface, (X, Y)=(100,100) represent the top right corner of the surface, and (X, Y)=(50,50) shows the center of the surface, for example. The spatial cardio-respiratory map 1410 is a bipolar ECG configuration where Lead I is the left arm (LA) and right arm (RA) lead, Lead II is the left leg (LL) and RA lead, and Lead III is the LL and LA lead. The spatial cardio-respiratory map 1410 can also be used to determine a unipolar ECG configuration, where a VR=RA-0.5* (LA+LL), aVL=LA-0.5* (RA+LL), and aVF=LL-0.5* (RA+LA). The spatial cardio-respiratory map 1420 is a pericardial ECG configuration using V1, V2, V3, V4, V5, and V6.

The method 1300 includes creating 1340 cardiac specific or respiratory specific combinations. The cardiac specific or respiratory specific combinations are generated from the MSMDA data using the surface location map and the spatial cardio-respiratory maps. The resulting combinations provide the closest match to the reference spatial cardio-respiratory map given the location of the sensors in the surface location map. The output will be a set of optimized combinations of the input MSMDA data. A process similar to that described for FIG. 9 in U.S. patent application Ser. No. 16/595,848, filed Oct. 8, 2019, can be used, the entire disclosure of which is hereby incorporated by reference. Other techniques can be used for joint processing as is known to those of skill in the art. Referring back to FIG. 14, each of the configurations can be mapped into a combination of the MSMDA data as represented by the surface location map. For example, in order to reconstruct Lead I, sensor combinations which represent RA and LA are needed. Using the shown map, RA is a function of the two left sensors 1432 and 1438 at (X, Y)=(0,0) and (X, Y)=(0,100). Similarly, LA is a function of the two right sensors 1434 and 1436 at (X, Y)=(100,0) and (X, Y)=(100,100). Other combinations are possible.

The method 1300 includes generating 1350 synthetic time series and sound streams. The cardiac specific or respiratory specific combinations of the MSMDA data are used to generate the synthetic cardio-respiratory time series and sound streams. In an implementation, each cardiac specific or respiratory specific combination can be processed independently using the method 700. In an implementation, the cardiac specific or respiratory specific combinations can be processed jointly to provide enhanced synthetic time series and sound streams since the MSMDA data may be correlated and may each partially capture the cardio-respiratory information. In this case, the method 700 is updated to accommodate joint pre-processing of MSMDA data. For example, this can be done using the relationship analysis described with respect to FIG. 9 in U.S. patent application Ser. No. 16/595,848, filed Oct. 8, 2019, can be used, the entire disclosure of which is hereby incorporated by reference. Other techniques can be used for joint processing as is known to those of skill in the art.

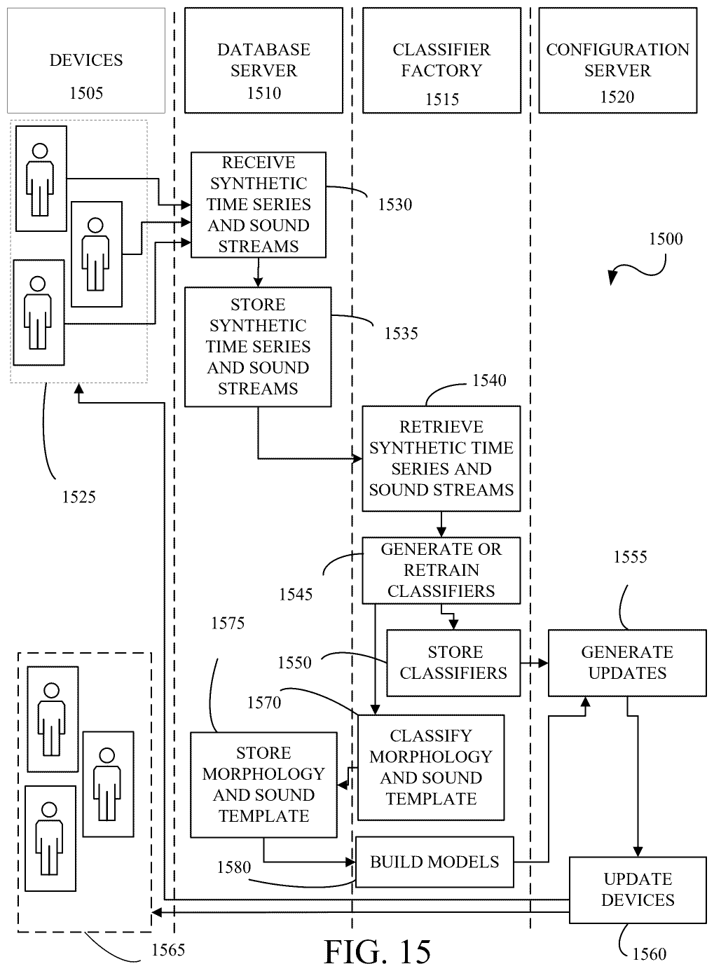

FIG. 15 is a swim lane diagram 1500 for building individualized and population morphology templates and generating classifiers for new devices or refreshing classifiers for existing devices. The swim lane diagram 1500 includes devices 1505 which include a first set of devices 1525 and a second set of devices 1565, a database server 1510, classifier factory 1515, and a configuration server 1520. In an implementation, the database server 1510, the classifier factory 1515, and the configuration server 1520 can be implemented at computing platform 420, for example.

The first set of devices 1525 generate synthetic time series and sound stream data which are received (1530) and stored (1535) by the database server 1510. The classifier factory 1515 retrieves the synthetic time series and sound stream data (1540) and generates or retrains classifiers using the synthetic time series and sound stream data (1545). The generated or retrained classifiers are stored by the classifier factory 1515 (1550). The generated or retrained classifiers are used by the classifier factory 1515 to classify morphology and sound templates (1570) into normal, abnormal, and like categories to automatically detect different arrhythmias or diseases, for example. The morphology and sound templates are stored (1575) in the database server 1510. In an implementation, the database server 1510 can include one or more databases for each morphology or sound type. For example, a database for normal morphologies and a separate database for each condition or disease. In an implementation, the storing of the synthetic time series and sound stream data (1535) and the morphology and sound templates (1575) can be the same database or different databases. The configuration server 1520 obtains the generated or retrained classifiers and generates an update (1555) for devices 1525. The configuration server 1520 sends the update (1560) to both the first set of devices 1525 and to the second set of devices 1565, where the second set of devices 1565 may be new devices. This system can be used to retrain classifiers on old devices (such as the first set of devices 1525) as more data input is available from more devices 1505. The system can also be used to provide software updates with improved accuracy and can also learn personalized patterns and increase personalization of classifiers or data.

Implementations of controller 200, controller 214, processor 422, and/or controller 414 (and the algorithms, methods, instructions, etc., stored thereon and/or executed thereby) can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors or any other suitable circuit. In the claims, the term “controller” should be understood as encompassing any of the foregoing hardware, either singly or in combination.

Further, in one aspect, for example, controller 200, controller 214, processor 422, and/or controller 414 can be implemented using a general purpose computer or general purpose processor with a computer program that, when executed, carries out any of the respective methods, algorithms and/or instructions described herein. In addition or alternatively, for example, a special purpose computer/processor can be utilized which can contain other hardware for carrying out any of the methods, algorithms, or instructions described herein.

Controller 200, controller 214, processor 422, and/or controller 414 can be one or multiple special purpose processors, digital signal processors, microprocessors, controllers, microcontrollers, application processors, central processing units (CPU)s, graphics processing units (GPU)s, digital signal processors (DSP)s, application specific integrated circuits (ASIC)s, field programmable gate arrays, any other type or combination of integrated circuits, state machines, or any combination thereof in a distributed, centralized, cloud-based architecture, and/or combinations thereof.

The word “example,” “aspect,” or “embodiment” is used herein to mean serving as an example, instance, or illustration. Any aspect or design described herein as using one or more of these words is not necessarily to be construed as preferred or advantageous over other aspects or designs. Rather, use of the word “example,” “aspect,” or “embodiment” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1-28. (canceled)

29. A method comprising:
obtaining, from at least two sensors configured to be mounted on a bed, ballistocardiogram (BCG) data from the at least two sensors, wherein the at least two sensors are configured to capture BCG data for one or more subjects in relation to a substrate configured to support the one or more subjects;
for each of the one or more subjects:
pre-processing the captured BCG data to obtain cardio-respiratory BCG data;
sub-sampling the cardio-respiratory BCG data to generate the cardio-respiratory BCG data at a cardio-respiratory sampling rate conducive to cardio-respiratory signal generation;
cardio-respiratory processing the sub-sampled cardio-respiratory BCG data to generate a cardio-respiratory parameter set;
generating a synthetic cardio-respiratory signal from at least the cardio-respiratory parameter set and a cardio-respiratory event morphology template;
determining a condition of the subject based on the synthetic cardio-respiratory signal; and
transmitting at least one of the condition and the synthetic cardio-respiratory signal to a remote user associated with the subject.

30. The method of claim 29, wherein the condition comprises a heart condition of at least one of atrial fibrillation, atrial flutter, ventricular fibrillation, ventricular flutter, a bundle branch block, valve stenosis, myocardial ischemia, and myocardial infarction.

31. The method of claim 29, wherein the condition comprises a breathing condition of at least one of apnea, hypopnea, Cheyne-Stoke breathing, and snoring.

32. The method of claim 29, wherein the at least two sensors comprise at least four load cells configured to be mounted on the bed, each load cell configured to be positioned to intervene in load transferred from the substrate to a floor.

33. The method of claim 32, wherein each load cell is configured to be positioned in the substrate to intervene in load transferred from the substrate to the floor.

34. The method of claim 29, wherein the substrate is configured to be coupled to a frame having multiple legs, and wherein the at least two sensors are configured to be coupled to the multiple legs.

35. The method of claim 29, wherein the at least two sensors are configured to be subject contactless.

36. The method of claim 29, wherein pre-processing the captured BCG data comprises filtering the captured BCG data based on proximity of a first of the at least two sensors to the substrate relative to proximity of a second of the at least two sensors to the substrate.

37. The method of claim 29, further comprising:
training a classifier based on the cardio-respiratory BCG data to generate at least a cardio-respiratory morphology classifier and a sound stream classifier; and
making classifications on non-classified cardio-respiratory BCG data using at least one of the cardio-respiratory morphology classifier and the sound stream classifier.

38. The method of claim 37, further comprising:
updating one or more other classifiers using at least one of the cardio-respiratory morphology classifier and the sound stream classifier.

39. A system comprising:
a bed including a substrate, the substrate configured to support one or more subjects;
at least two sensors configured to be mounted on the bed, the at least two sensors configured to capture ballistocardiogram (BCG) data from subject actions with respect to the substrate; and
a processor configured to be in data communication with the at least two sensors, the processor configured to:
pre-process the captured BCG data to obtain cardio-respiratory BCG data;
sub-sample the cardio-respiratory BCG data to generate the cardio-respiratory BCG data at a cardio-respiratory sampling rate conducive to cardio-respiratory signal generation;
cardio-respiratory process the sub-sampled cardio-respiratory BCG data to generate a cardio-respiratory parameter set;
generate a synthetic cardio-respiratory signal from at least the cardio-respiratory parameter set and a cardio-respiratory event morphology template;
determine a condition of the subject based on the synthetic cardio-respiratory signal; and
transmit at least one of the condition and the synthetic cardio-respiratory signal to a remote user associated with the subject.

40. The system of claim 39, wherein the condition comprises a heart condition of at least one of atrial fibrillation, atrial flutter, ventricular fibrillation, ventricular flutter, a bundle branch block, valve stenosis, myocardial ischemia, and myocardial infarction.

41. The system of claim 39, wherein the condition comprises a breathing condition of at least one of apnea, hypopnea, Cheyne-Stoke breathing, and snoring.

42. The system of claim 39, wherein the at least two sensors comprise at least four load cells configured to be mounted on the bed, each load cell configured to be positioned in the bed to intervene in load transferred from the substrate to a floor.

43. The system of claim 42, wherein each load cell is configured to be positioned in the substrate to intervene in load transferred from the substrate to the floor.

44. The system of claim 39, wherein the substrate is configured to be coupled to a frame having multiple legs, and wherein the at least two sensors are configured to be coupled to the multiple legs.

45. The system of claim 39, wherein when pre-processing the captured BCG data, the processor is configured to filter the captured BCG data based on a proximity of a first of the at least two sensors to the substrate relative to a proximity of a second of the at least two sensors to the substrate.

46. The system of claim 39, wherein the processor is further configured to:
train a classifier based on the cardio-respiratory BCG data to generate at least a cardio-respiratory morphology classifier and a sound stream classifier; and
make classifications on non-classified cardio-respiratory BCG data using at least one of the cardio-respiratory morphology classifier and the sound stream classifier.

47. The method of claim 46, wherein the processor is further configured to:
update one or more other classifiers using at least one of the cardio-respiratory morphology classifier and the sound stream classifier.

48. A method comprising:
obtaining, from at least two sensors configured to be mounted on a bed in spaced-apart relation, ballistocardiogram (BCG) data from the at least two sensors, wherein the at least two sensors are configured to capture BCG data for one or more subjects in relation to a substrate configured to support the one or more subjects;
for each of the one or more subjects:
pre-processing the captured BCG data to obtain cardio-respiratory BCG data, including filtering the captured BCG data based on a proximity of a first of the at least two sensors to the substrate relative to a proximity of a second of the at least two sensors to the substrate;
sub-sampling the cardio-respiratory BCG data to generate the cardio-respiratory BCG data at a cardio-respiratory sampling rate conducive to cardio-respiratory signal generation;
cardio-respiratory processing the sub-sampled cardio-respiratory BCG data to generate a cardio-respiratory parameter set;
generating a synthetic cardio-respiratory signal from at least the cardio-respiratory parameter set and a cardio-respiratory event morphology template;
determining a condition of the subject based on the synthetic cardio-respiratory signal, wherein the condition comprises at least one of a heart condition and a breathing condition; and
transmitting at least one of the condition and the synthetic cardio-respiratory signal to a remote user associated with the subject.
